(19) United States
(12) Patent Application Publication
(10) Pub. No.: US 2021/0092919 A1
(43) Pub. Date: Apr. 1, 2021

(54) WATERING SYSTEM

(71) Applicants: Alexander Zehnbacht, West Linn, OR (US); Kevin James King, Vancouver, WA (US)

(72) Inventors: Alexander Zehnbacht, West Linn, OR (US); Kevin James King, Vancouver, WA (US)

(73) Assignee: Dripio, LLC, West Linn, OR (US)

(21) Appl. No.: 16/586,174

(22) Filed: Sep. 27, 2019

(51) Int. Cl. A01G 25/16; H04B 3/50; H04L 12/40; H04L 12/403; G01N 33/24; G01F 15/06

(52) U.S. Cl. CPC A01G 25/167; H04B 3/50; H04L 12/40045; H04L 12/40019; H04L 2012/4026; H04L 12/403; G01N 33/246; G01F 15/063; H04L 12/40039

(57) ABSTRACT

The present invention relates generally to agriculture and irrigation of plants. A watering system comprising a master unit and one or more node units are able to carry out bidirectional communication via a power control bus, which may be a coaxial cable. The power control bus provides power and data communication to one or more node units. The one or more node units are configured to receive, carry out actions, or respond to queries from a master unit. Actions that may be taken by one or more node units may include the operation of irrigation valves and query of data related to moisture or other chemistry of a plant growing soil. A master unit may connect to an internet cloud via Wifi or other communication method, wherein a watering system of the present invention may be operable based on at least one data from an internet cloud.

### RELATED APPLICATIONS

This application claims the benefit of U.S. Provisional Patent Application No. 62/738,667 filed Sep. 28, 2018, entitled WATERING SYSTEM and U.S. Provisional Patent Application No. 62/794,706 filed Jan. 21, 2019, entitled COAXIAL CONNECTING APPARATUS and U.S. Provisional Patent Application No. 62/802,205 filed Feb. 6, 2019, entitled DOSED IRRIGATION SYSTEM AND METHOD and U.S. Provisional Patent Application No. 62/884,017 filed Aug. 7, 2019, entitled WATERING SYSTEM and incorporates the disclosures of those applications in their entirety by reference.

### BACKGROUND

Some irrigation systems may utilize individual watering zones. Smart watering (sprinkler) controllers are connected to a set number of solenoid valves, each controlling a separate watering line usually buried in the ground. The line is then terminated with watering devices such as sprinklers. Each zone typically waters an area of a lawn and/or a garden. A zone can be converted to drip line, however each system by design is limited to a maximum number of zones, each new zone requiring piping installation from the solenoid valve box to the area being watered.

### OVERVIEW

Some embodiments described herein may feature individualized control of watering of assorted residential garden plants on a shared watering line. Some embodiments may use a smart cloud-assisted watering controller connected with a coaxial wire to a network of Node Units (e.g., smart valves) delivering water to an individual plant or group of plants via shared drip irrigation tubing.

In some embodiments, a wire (such as a coaxial cable) may serve as both a DC power line and a data bus. The controller may be a Master and controls the communication with Node Units (Slaves) using a collection of local addresses. A new Slave being added to the bus is assigned a new address. Node Units may be designed to connect in daisy chain. Some embodiments may support splitting of the wire line, as well.

Some embodiments may feature one or more of the following:

1) A capacitor is charged via a diode in each Node Unit. This capacitor may be used to power a processor of a Node Unit during brief losses of power.

2) Power to the system may be momentarily interrupted. A software of a processor of a Node Unit may interpret the momentary interruption of power as a bit of data, for example a “1” or a “0”. A software of a processor may interpret non-interruption of power as a bit of data, for example, a “0”, or a “1”.

3) Power to the system may be interrupted or not interrupted over a period of intervals of time. A software of a processor of a Node Unit may sample power to the system over a period of intervals of time, and may interpret the interruption or non-interruption of power to be a sequence of “1” or “0” bits, and may assemble a sequence of “1” or “0” bits into a memory, wherein the sequence in memory may comprise a byte of data.

4) A software of a processor of a Node Unit may interpret one or more of a byte of data as one or more of an address, a command, an argument, an unused data, a checksum, or any combination or sequence thereof, and may cause an electrical signal to be sent to one or more of an electrically operated valve to cause the valve to open or to shut based on the content of one or more of a byte of data.

5) The center conductor of a coax cable may be used to carry said power to the system, and the outer conductor of a coax cable may be used as a ground or return path of said power to the system, or vise versa.

6) Other examples of two-conductor cabling can be used to embody a substantially similar system.

7) If a wire or wires or a cable has three or more conductors, power may be provided via a pair of conductors, and data may be sent one direction or both directions via one or more additional conductors of a wire or cable.

In some embodiments, a wire, or plurality of wires, or cable such as a coaxial cable, may reliably provide both power and data. In view of the fact that a connection between the main controller and the cloud might be interrupted for periods of time, in some embodiments the main controller may be cloud-assisted yet cloud-independent, meaning the watering is scheduled and queued in the controller itself, so it can water independently of cloud for at least a set period of time (say a week or so). The power supply may be internal or external to the main controller (in some embodiments, the power supply may be provided in a separate waterproof enclosure outside of a main controller enclosure, and connect both using IP67 waterproof DC connectors).

### BRIEF DESCRIPTION OF THE DRAWINGS

A more complete understanding of the present invention may be derived by referring to the detailed description when considered in connection with the following illustrative figures. In the following figures, like reference numbers refer to similar elements and steps throughout the figures.

FIG. 1 representatively illustrates a simplified diagram of a watering system, according to various embodiments.

FIG. 2 representatively illustrates a simplified diagram of a Master Unit, according to various embodiments.

FIG. 3 representatively illustrates a simplified diagram of a Node Unit, according to various embodiments.

FIG. 4 representatively illustrates a simplified diagram of a circuitry of a Master Unit, according to various embodiments.

FIG. 5 representatively illustrates a simplified diagram of a power distribution arrangement of a Master Unit, according to various embodiments.

FIG. 6 representatively illustrates a simplified diagram of a power distribution arrangement of a Node Unit, according to various embodiments.

FIG. 7 representatively illustrates a simplified electronic schematic diagram of a switching circuitry of a Master Unit, according to various embodiments.

FIG. 8 representatively illustrates a simplified electronic schematic diagram of a bus interface circuitry of a Node Unit, according to various embodiments.

FIG. 9 representatively illustrates a simplified electronic schematic diagram of a latching valve control circuitry of a Node Unit, according to various embodiments.

FIG. 10 representatively illustrates a simplified diagram of a wave form of a power control bus, according to various embodiments.

FIG. 11 representatively illustrates a simplified diagram of a byte transmission waveform on a power control bus, according to various embodiments.

FIG. 12 representatively illustrates a simplified diagram of a packet transmission waveform having a transmit period and a reply period on a power control bus, according to various embodiments.

FIG. 13 representatively illustrates a simplified diagram of a packet structure of the present invention on a power control bus, according to various embodiments.

FIG. 14 representatively illustrates a simplified flow chart of a communication with response between a master unit and a node unit, according to various embodiments.

FIG. 15 representatively illustrates a simplified flow chart of a communication between a master unit, a network or cloud, a remote server, and another device such as a smart phone, according to various embodiments.

### DETAILED DESCRIPTION OF DRAWINGS

Turning now to the drawings, several exemplary embodiments are described. One skilled in the art of electronic design, power systems, irrigation systems, and plumbing systems will be able to understand and create the various embodiments. Though an embodiment is suggested, it will be clear that other embodiments are possible without deviating from the spirit of the present invention.

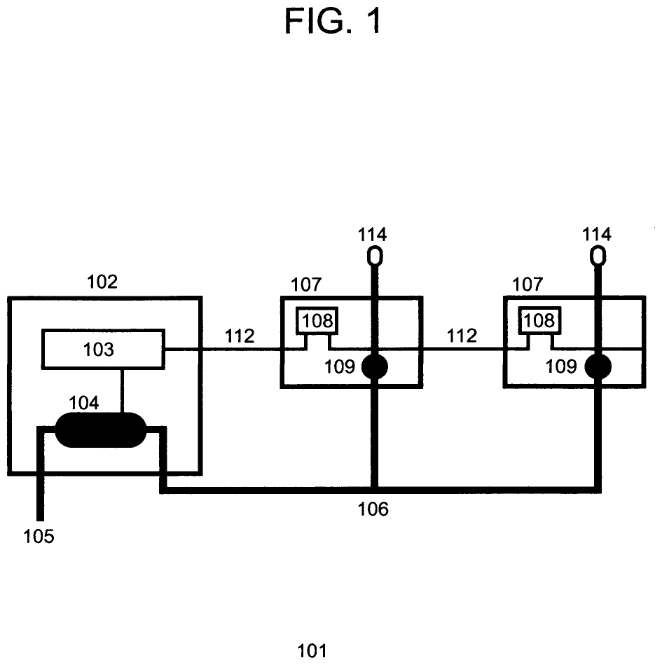

FIG. 1 may illustrate a perspective view of a watering system 101, according to various embodiments. A master unit 102 may comprise a circuitry 103 and a plumbing assembly 104. A plumbing assembly may receive water, fluid, or nutrients or any combination thereof from a water source 105, which may be for example a household garden hose spigot. A plumbing assembly 104 may supply water to a distribution header 106, which may carry water to one or more of a Node Unit 107.

A circuitry 103 may be electrically coupled to various elements of a plumbing assembly 104 to control or monitor various aspects of the fluid flow of a plumbing assembly 104. A circuitry 103 may provide electrical power to a power control bus 112, which may be electrically connected to a circuitry 108 of one or more of a node unit 107. A plurality of a node unit 107 may be connected by daisy chaining a power control bus 112 between them.

A node unit 107 may comprise a circuitry 108 that is powered via electrical power from a power control bus 112. A circuitry 108 may be electrically coupled to a valve 109, such as for example, a latching or non-latching solenoid valve. When a valve 109 is opened, a fluid may be allowed to flow from a distribution header 106 to a watering device 114. A watering device 114 may be for example, but not limited to a drip nozzle, misting nozzle, soaker hose, or a second header, tube, or hose that may deliver a fluid to another watering device as described. When a valve 109 is shut, it may substantially stop the flow of a fluid to a watering device 114.

A circuitry 103 of a master unit 102 may transmit coded control data or instructions via a power control bus 112 to the circuitry 108 of one or more of a node unit 107. A power control bus 112 may be a two conductor cable, such as but not limited to common twisted pair electrical wire, low voltage lighting power cable, or coaxial cable such as for example RG-59 cable, RG-6 cable or the like. This coaxial cable is commercially available and commonly used to route satellite and cable television signals in residential buildings. One advantage of using coaxial cable is that waterproof connectors are readily available commercially and the cable is difficult to connect incorrectly. There is minimal concern of a user connecting a coaxial cable with the polarity reversed.

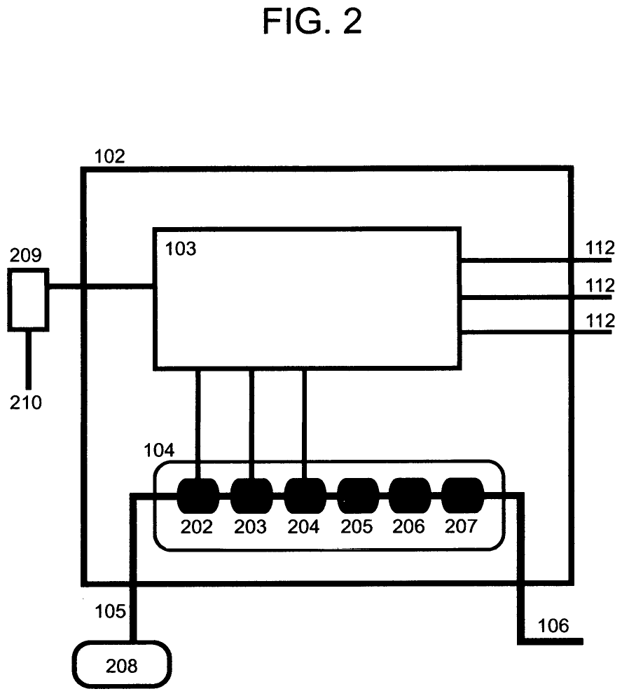

FIG. 2 may representatively illustrate a simplified diagram of a master unit 102, according to various embodiments. A master unit 102 may comprise a circuitry 103 that is powered via a power source 209 such as a power regulator, which in turn may draw power from another source 210 such as a common household power outlet. A plumbing assembly 104 of a master unit 102 may comprise a variety of piping or plumbing parts. In one exemplary embodiment, a plumbing assembly 104 may comprise a valve 202, a pressure sensor 203, a flow meter 204, a filter 205, a pressure reducer 206, and a backflow preventer 207, or any combination or arrangement thereof. A valve 202 may be an electrically operated valve such as for example a latching or non-latching solenoid valve. Some components of a plumbing assembly 104 may be electrically controlled, or may provide electrical connections for sensors and the like. These electrical parts of a plumbing assembly 104 may be electrically connected to a circuitry 103. A circuitry 103 of a master unit 102 may connect to one or more of a power control bus 112.

A plumbing assembly 104 may connect 105 to a fluid source 208, such as for example but not limited to, a household garden hose spigot. When a valve 202 is open, a fluid from a source 208 may be allowed to flow through one or more components of a plumbing assembly 104 to a distribution header 106.

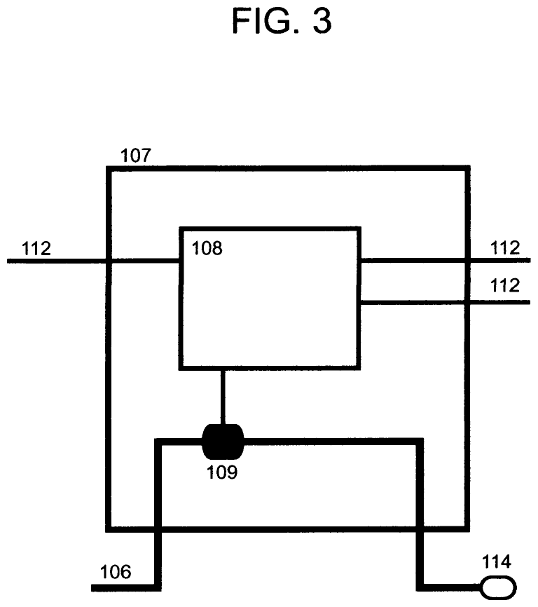

FIG. 3 may representatively illustrate a simplified diagram of a node unit 107, according to various embodiments. A node unit 102 may comprise a circuitry 108 that is powered via a power control bus 112. The connecting point of a power control bus 112 to a circuitry 108 may also electrically connect to one or more additional connecting points where a power control bus may be daisy chained between a plurality of a node unit 107. A circuitry 108 may be electrically connected to a valve 109 such as an electrically controlled latching or non-latching solenoid valve. A valve 109 may connect to a distribution header 106. When a valve is open, a fluid of a distribution header 106 may flow to one or more of a watering device 114.

In various embodiments, a circuitry 108 of a node unit 107 may also connect to other electrically controlled plumbing components or sensors, as well as other accessory components such as but not limited to nutrient distribution systems, lighting devices such as landscape lighting, various sensing systems such as soil moisture sensors, additional flow meter devices, or other monitoring devices such as digital cameras, motion sensors and the like, and may also connect to other accessory devices such as sound, air, or movement production devices that may be used to deter wildlife from an area. Data, control signals, data signals and the like may be communicated between various combinations of the various devices described via a power control bus 112 between a node unit 107 and a master unit 102.

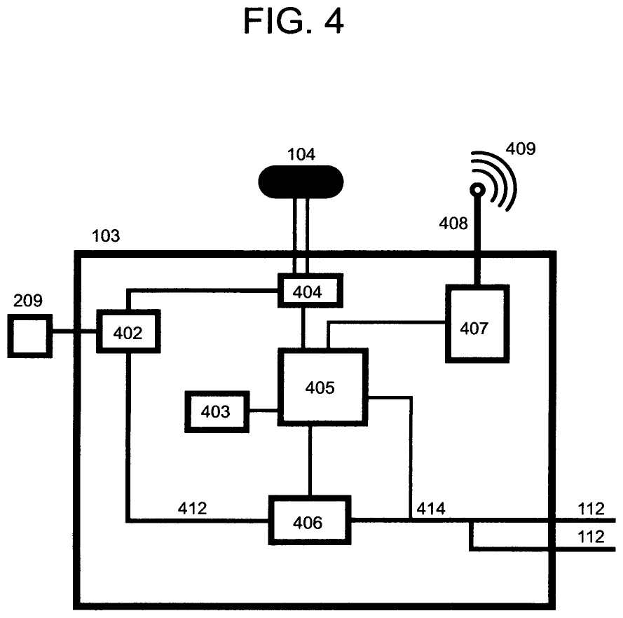

FIG. 4 may representatively illustrate simplified diagram of a circuitry 103 of a master unit 102, according to various embodiments. A circuitry 103 may be assembled on one or more of a printed circuit board or similar assembly as would be known to one skilled in the art.

A circuitry 103 may receive power from a power source 209. A power source 209 may supply direct current power at 12 volts for example, though other voltages or alternating current power may be used in various embodiments as applicable and as would be known to one skilled in the art. Power may pass through various power management or regulation components 402 which may include various switching or non-switching regulators or monitoring circuitry. Power may be delivered via a power connection 412 to a switching circuitry 406 which may control power to a power control bus 112. Regulation components 402 may deliver power to a valve control circuit 404 which may be used to operate a valve 104. A valve control circuit 404 may also or alternately be connected directly to a power source 209.

A microprocessor 405 may be operably coupled to various components of a circuitry 103, and may draw power from regulation components 402, a power source 209, or other power source. A microprocessor 405 may be operably coupled to various supporting components 403 such as crystals, resonators, non-volatile memory such as an EEPROM device, or other components useful in a given embodiment. A microprocessor 405 may be coupled 414 to a power control bus 112 directly or via other components such that a voltage of a power control bus 112 may be monitored via a microprocessor 405.

A radio communication device 407 may comprise or be coupled to an antenna 408, which may transmit or receive radio signals 409. A radio communication device 407 may be a WiFi module, Bluetooth module, XBee device, or any other means of communicating radio signals to a microprocessor. In various embodiments, the functionality of a microprocessor 405, supporting components 403, and radio communication device 407 may be comprised within a single module or any combination thereof. A radio communication device 407 may allow a circuitry 103 to wirelessly communicate with a WiFi access point or other communication or networking device which may provide access to various information systems, databases, control systems, operational programs and the like which may be on a network, a computer or server, a smart phone, the internet, a different master unit 102 or the like, or any combination thereof.

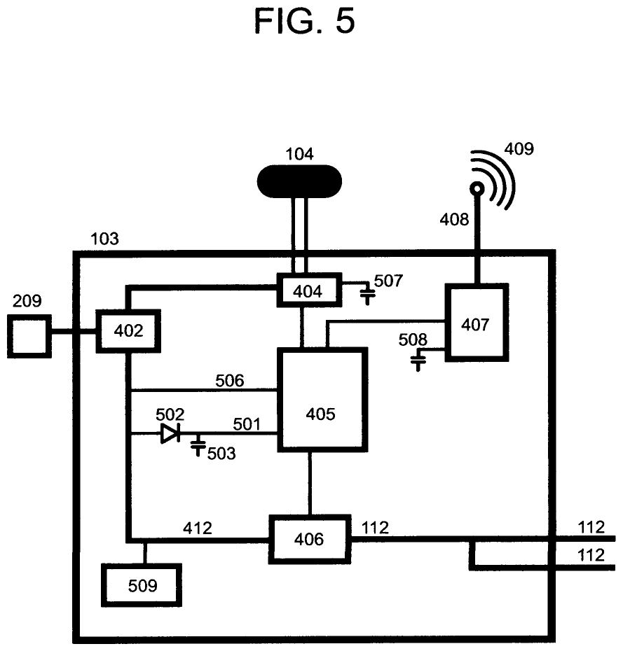

FIG. 5 may representatively illustrate a simplified diagram of a power distribution arrangement of a master unit 102, according to various embodiments. A microprocessor 405 may be powered via a power source 501 which is also powered by a capacitor 503 or super capacitor or similar device capable of storing energy, which may remain charged via a diode 502. This arrangement may allow a microprocessor 405 to continue operating for a period of time in the event power is lost to a master unit 102. A battery backup, solar cell, or other supplemental power source 509 may be provided. A radio communication device 407 may be powered via a capacitor 508 in a similar way. A valve control circuit 404 may comprise or be powered by a capacitor 507 in a similar way. A capacitor 507 may be sized appropriately so as to provide enough energy to operate a valve 104 in the event of a loss of power to other parts of a circuitry 103.

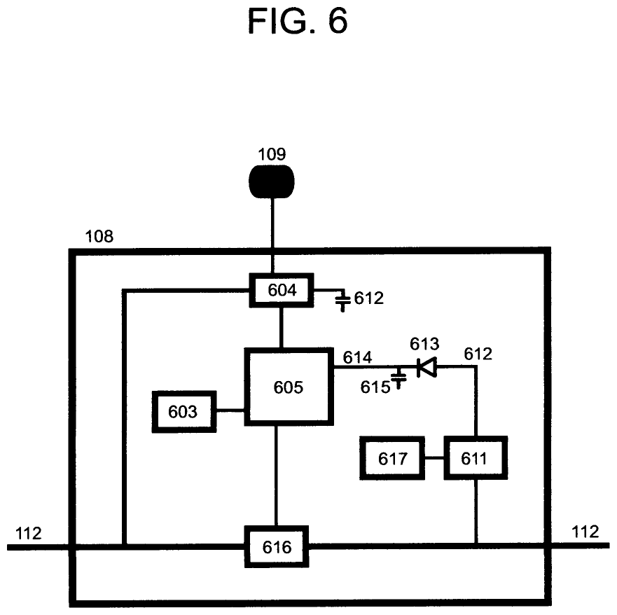

FIG. 6 may representatively illustrate a simplified diagram of a power distribution arrangement of a node unit 108, according to various embodiments. A microprocessor 605 may be powered via a power source 614 which may also powered by a capacitor 615 or super or super capacitor or similar device capable of storing energy, which may remain charged via a diode 613. A microprocessor 605 may be powered via regulation components 611, as well as optionally a battery backup, solar cell, or other supplemental power source 617. A capacitor 615 may be sized appropriately so as to provide enough energy to operate a microprocessor 605 during periods brief power loss to a power control bus 112. A microprocessor 605 may be operably coupled to various supporting components 603 such as crystals, resonators, non-volatile memory such as an EEPROM device, or other components useful in a given embodiment. A valve control circuit 604 may comprise or be powered by a capacitor 612 in a similar way. A capacitor 612 may be sized appropriately so as to provide enough energy to operate a valve 109 in the event of a loss of power to other parts of a circuitry 108. A microprocessor 605 may be operably coupled to bus interface circuitry 616 which may allow a microprocessor 605 to measure or otherwise interact with a power control bus 112.

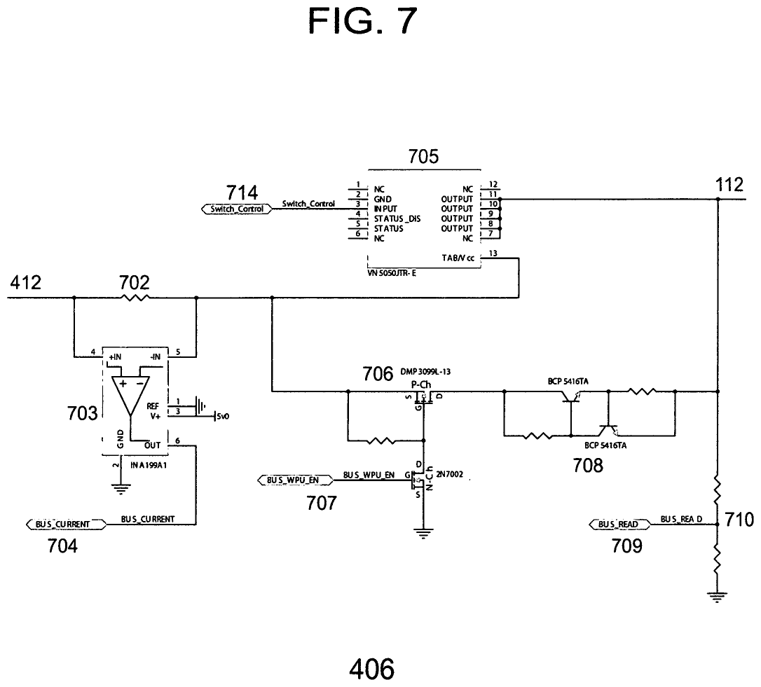

FIG. 7 may representatively illustrate a simplified electronic schematic diagram of a switching circuitry 406 of a master unit 102, according to various embodiments, and may be described as follows. A power connection 412 may provide power to a switching circuitry 406. Electric current may pass through a resistor 702 which may be monitored by an amplifier 703 such that current through a resistor 702 may be monitored. An analog signal 704 may be provided to a microprocessor 405, wherein an analog signal 704 may change proportional to current flow of a resistor 702. A microprocessor 405 may monitor a signal 704 and may stop the flow of current via a switching element 705 if current exceeds a threshold. A switching element 705 may be controlled via a signal 714 via a microprocessor 405. When a switching element 705 is turned on, electric current may be allowed to energize a power control bus 112 to a voltage near the voltage of a power source 209, for example, approximately 12 volts.

A weak pull-up circuit may be enabled by turning on a mosfet 706 via a control signal 707 which may connect to a microprocessor 405, which may in turn energize a power control bus 112. This weak pull-up circuit may power a power control bus 112 via a resistor, or via a current limiting component or circuit 708. In certain operational conditions, for example, when a response is expected from a node unit 107, a switching element 705 may be turned off, and a mosfet 706 may be turned on so as to energize a power control bus 112 via the current limiting component 708. This may allow a circuitry 108, 616, of a node unit 107 to pull the voltage of a power control bus 112 to a lower level, which may be observed or measured by a microprocessor 405 via a signal 709. A signal 709 may be provided by a voltage divider 710 which may reduce the voltage of a power control bus 112 to a lower level which may be within an acceptable voltage limit of a microprocessor 405. When a circuitry 108 of a node unit 107 sinks current of a power control bus 112, when a current limit of a current limiting component 708 is reached, the voltage of a power control bus 112 may be lowered and monitored via signal 709 by a microprocessor 405.

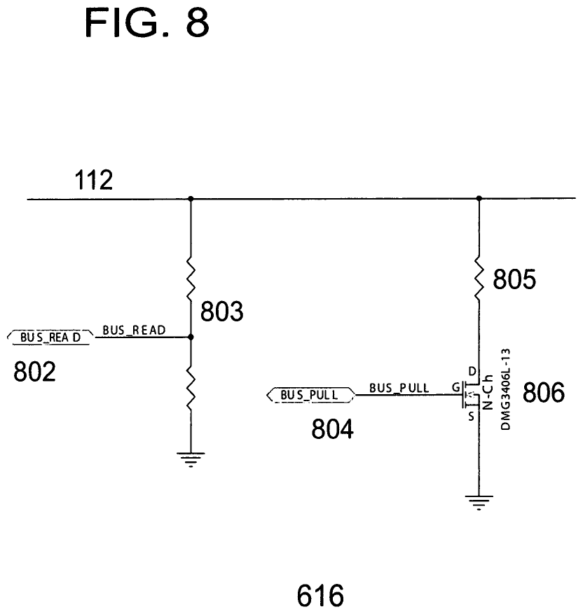

FIG. 8 may representatively illustrate a simplified electronic schematic diagram of a bus interface circuitry 616 of a node unit 107, according to various embodiments. Various interface circuitry 616 may connect to a power control bus 112. For example, a signal 802 may be connected to a microprocessor 605 either directly or via other components for example a voltage divider 803. Signal 802 may allow a microprocessor 605 to measure voltage on a power control bus 112. A mosfet 806 or similar device may be controlled via a signal 804 from a microprocessor 605, such that electric current may sink from a power control bus 112 directly through a mosfet 806 or via a resistor 805, which may cause a voltage of a power control bus 112 to be lower when a mosfet 806 is turned on. A resistor 805 may be a relatively low resistance value and have a high wattage capacity such that maximum current limits of mosfet 806 are always respected.

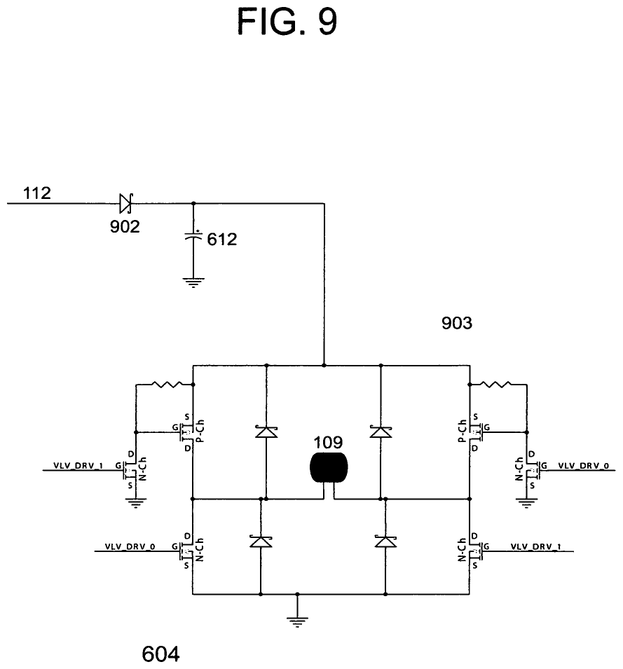

FIG. 9 may representatively illustrate a simplified electronic schematic diagram of a latching valve control circuitry 604 of a node unit, according to various embodiments. Though not illustrated, it is noted that a valve control circuit 404 of a circuitry 103 of a master unit 102 may be substantially similar to a latching valve control circuitry 604. It is one advantageous aspect of latching valve control circuitry 604 that at least a portion of a the energy required to operate a valve 109 be stored in a capacitor 612 or other component capable of storing energy even if power is lost or temporarily lost to a power control bus 112. A latching valve control circuitry 604 may be powered via a power control bus 112 or other component, regulator, or power source electrically connected to a circuitry 108, and may be powered through a diode 902, such that a capacitor 612 remains substantially charged for a period of time following a loss of power or voltage of a power control bus 112. Those skilled in the art of electronics may recognize portions of the circuit to comprise an “H-Bridge” circuit, this circuit is commonly used by those skilled in the art of electronics to provide a flow of current in one direction or the opposite direction depending on at least two control signals. Signals VLV_DRV_0 905 and VLV_DRV_1 906 may be connected to a microprocessor 605. When signal 905 is high and signal 906 is low, a latching valve 109 may operate in a given direction, causing the valve 109 to actuate open or shut, depending on polarity. When signal 906 is high and signal 905 is low, a latching valve 109 may operate in the opposite direction. When either signal 905 or 906 is switched to a high state by a microprocessor 605, electrical energy or current may flow to the solenoid of a valve 109, the electrical energy may be sourced from a power control bus 112 directly or via a diode 902 or from a capacitor 612 or from any combination or proportion thereof.

As a power control bus 112 may comprise a long length of cable, for example 1000 feet or more, a long cable may provide increased resistance or impedance to the flow of electric current, which may cause a voltage drop near the input of a latching valve control circuitry 604 when current is allowed to flow to a valve 109. A capacitor 612 may store enough energy to actuate a valve 109 even given the limitations that may be introduced by a long length of cable between a master unit 102 and one or more of a node unit 107.

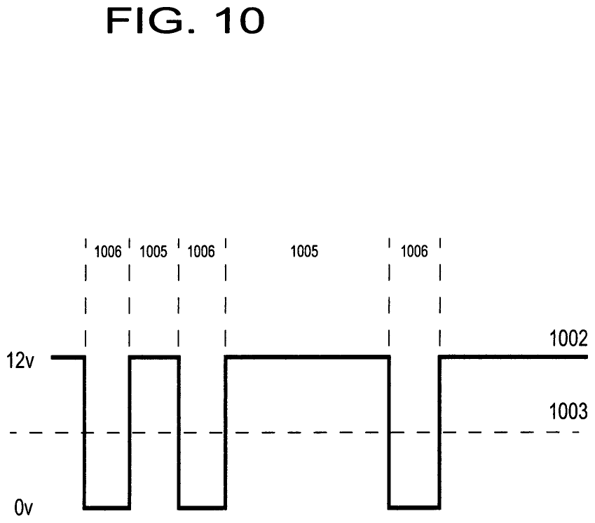

FIG. 10 may representatively illustrate a simplified diagram of a wave form 1002 of a power control bus 112, according to various embodiments. A wave form 1002 may illustrate a voltage vs. time plot, where a voltage is controlled from a high state, for example but not necessarily 12 volts, to a lower state which may be 0 volts or some other magnitude lower than the starting voltage. A threshold 1003 may be programmed into a port or pin peripheral or any other aspect of a programming of a microprocessor 405 or 605 or any combination thereof. A microprocessor 405 or 605 may sense or receive the voltage or representative voltage of a waveform 1002 by an interrupt triggered by changing the state of an input pin of a microprocessor, or an analog to digital fractionally if a microprocessor either automatically or as driven by a programming of a microprocessor. A voltage 1002 may generally exist at a higher voltage during a given period or periods 1005, and may occasionally exist at a lower voltage during a different period or periods 1006.

A voltage 1002 may be controlled by a programming of a microprocessor 405. For example, a microprocessor 405 via a signal 714 to a switching element 705 may energize a power control bus 112. A microprocessor 405 may cause a voltage of a control bus 112 may then be brought to a lower level by a microprocessor 405 via signal 714 may turn off a switching element 705, which may de-energize a power control bus 112 during a period 1006, and may as described above, re-energize a power control bus 112 during a subsequent period 1005 by turning back on a switching element 705 via signal 714. As a power control bus 112 may have some capacitance, a circuitry 103 may also comprise a circuit to assist in pulling the voltage 1002 low more quickly. This optional circuit may be similar to that of a node unit components 804, 805, 806. Using this described method, a programming of a microprocessor may control periods 1005 and 1006 of a voltage 1002 of a power control bus 112.

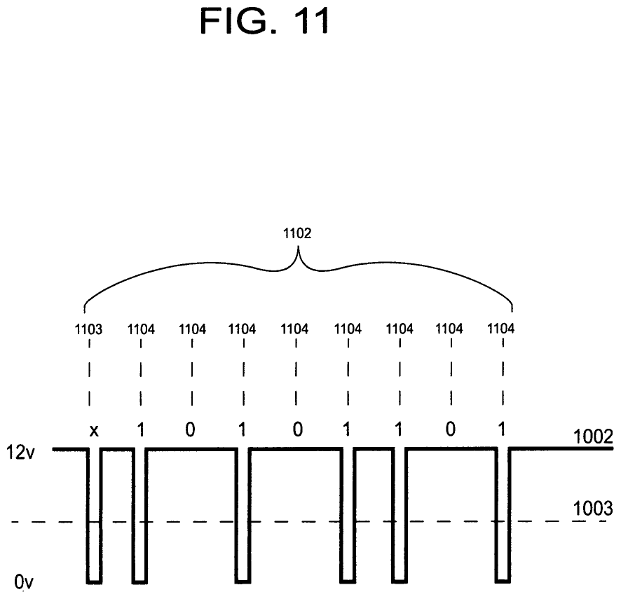

FIG. 11 may representatively illustrate a simplified diagram of a byte transmission 1102 waveform on a power control bus 112, according to various embodiments, and may be described as follows. Any embodiment described herein may transmit data using the following process.

Voltage 1002 to a power control bus 112 may be brought to a lower level at a starting point in time 1103. A different circuitry that is able to sense or read a voltage of a power control bus 112, for example but not limited to a circuitry 108 of a node unit 107, or a programming of a microprocessor comprised therein or otherwise operably connected to, may respond to a change of voltage to a level below that of a threshold 1003, said programming or other peripheral of a microprocessor may begin a timing interval.

A sequence of substantially predictable timing intervals may proceed. At each timing interval, or in response to a changing of voltage 1002 a microprocessor 605 of a circuitry 108 may shift in or set a memory of a bit or a 1 or a 0, in the present example, a 1 is shifted to a memory of a microprocessor 605. If a drop in voltage is sensed substantially coinciding with a timing interval 1104, a 1 is shifted to a memory, and if no drop in voltage is sensed substantially coinciding with a timing interval 1104, a 0 is shifted to a memory. To simplify operation and programming, a simple threshold 1003 may be used, a threshold 1003 may be set at any arbitrary value where a voltage 1002 is expected to be observable as being higher or lower than a threshold. A electronic comparator component may also be optionally used. Thus in this example, to simplify, a voltage may be reduced temporarily below a threshold which may be used as a timing marker to begin a substantially predictable sequence of timing intervals. This may be used to synchronize the timing of the reception of one or more bits of data between a master unit 102 and a node unit 107.

After a plurality of periods 1104, a sequence of 1's or 0's may be shifted into a memory of a microprocessor. In the example of FIG. 11, this sequence of 1's and 0's is 10101101. This may also be expressed in hexadecimal 0xAD. A similar method is able to send the binary representations of many kinds of data. The data may comprise control signals, such that via control signals a master unit 102 may control the operation of a node unit 107.

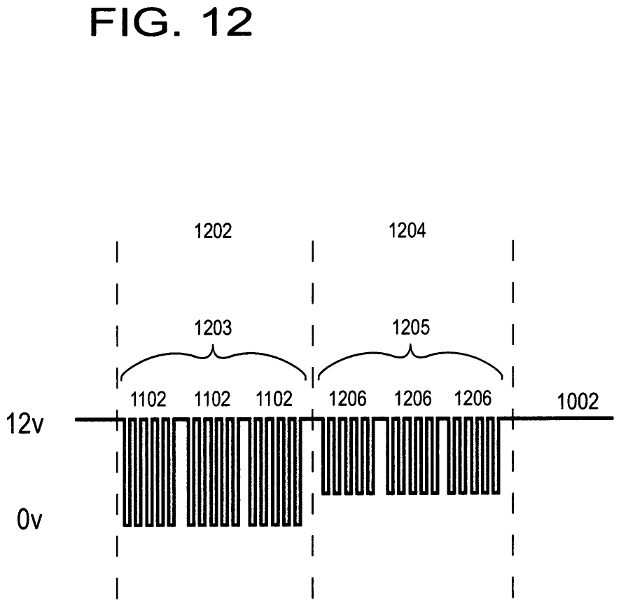

FIG. 12 may representatively illustrate a simplified diagram of a packet transmission waveform having a transmit period 1202 and a reply period 1204 on a power control bus 112, according to various embodiments. During a transmit period 1202, one or more byte transmissions 1102 may occur, wherein said one or more byte transmissions 1102 may comprise data from a master unit 102 to one or more node units 107. One or more of a byte 1102 in a sequence may be logically combined together by a programming of a microprocessor to form a transmit packet 1203. A subsequent reply period 1204 may allow a period of time, which may be substantially predictable, and for a circuitry 103 of a master unit 102 to configure to receive one or more of a response byte 1206 from a circuitry 108 of a node unit 107, and may allow for a circuitry 108 of one or more of a node unit 107 to configure to send one or more of a response byte 1206. One or more of a byte 1206 in a sequence may be logically combined together by a programming of a microprocessor to form a reply packet 1205.

During a response period 1204, a programming of a microprocessor 405 of a master unit 102 may configure at least one portion of a circuitry 103 as follows. A switching element 705 may be turned off via a signal 714. A weak pull up circuit 706 may be enabled via signal 707. During at least one portion of a response period 1204, a microprocessor 405 may read or sense the voltage of a power control bus 112 via a signal 709 directly or via other components 710. A microprocessor 405 may bit-shift one or more of a response bits into a memory using a similar method as may be illustrated in FIG. 11. One or more bits may be combined together to form one or more bytes in a similar way as may be illustrated in FIG. 11.

A response byte 1206 may be generated by a programming of a microprocessor 605 by turning on a current sink connected to a power control bus, for example, via 806 as controlled by signal 804, which may cause a voltage 1002 of a power control bus 112 to be lower. A timing of a response byte may be similar to that illustrated in FIG. 11.

It should be noted that a response byte may pull a voltage 1002 to a low level that is higher than that of a transmit byte. This difference in voltage may be caused by the voltage drop of current through a cable or resistor such as resistor 805 when a current sink 806 is turned on. During the time in which a circuitry 108 is pulling a voltage 1002 low, a voltage divider is essentially set up between weak pull up enable components 706, current limiting components 708, and sink resistor 805 and pull down components 806 (as well as any resistance present in the physical cable of a power control bus 112, which may increase with greater length). As would be known to one skilled in the art of electronics, appropriate values must be selected for said components so as to cause a voltage 1002 to reliably drop below a threshold when a sink 806 is turned on, while at the same time respecting the power dissipation limitations of the selected components at the given voltage and current flow.

It should be noted also that any electronic device that is drawing current from a power control bus 112 during a response period 1204 will cause the voltage 1002 to be reduced, which may negatively affect the headroom available to sense a voltage 1002 as being above or below a threshold. With this in mind, in some embodiments the circuitry 108 of all node units 107 connected to a power control bus 112 may be configured to enter a lower power configuration that is maintained during at least a portion of response period 1204. For example, a programming of a microprocessor 605 may turn off regulation components 611, any accessory components such as but not limited to lighting, indication lights, and the like, and a microprocessor 605 may enter a lower power state during a response period 1204.

In one exemplary embodiment having a plurality of node units 107 connected to a power control bus 112, every microprocessor 605 of every node unit 107 may configure every circuitry 108 to enter a lower power state during a response period 1204, even if the data 1102, 1203, 1205, 1206 may not pertain to the given node unit 107 or a specific node unit 107.

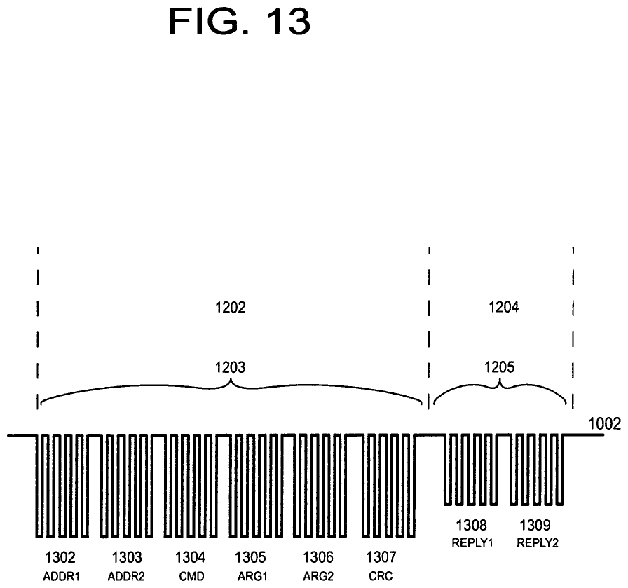

FIG. 13 may representatively illustrate a simplified diagram of a packet structure that may be utilized on a power control bus 112 in any embodiment described herein.

In one exemplary embodiment, a transmit packet 1203 may be transmitted during a transmit period 1202, and a reply packet 1205 may be communicated during a reply period 1204. A transmit packet 1203 may comprise six bytes 1102, wherein the first byte 1302 ADDR1 is eight bits of a sixteen bit address, and wherein the second byte 1303 ADDR2 is the remaining eight bits of a sixteen bit address, and wherein the third byte 1304 CMD is an eight bit value corresponding to a command, and wherein the fourth byte 1305 ARG1 is eight bits of a sixteen bit argument, and wherein a fifth byte 1306 ARG2 is the remaining eight bits of a sixteen bit argument, and wherein the sixth byte 1307 CRC is eight bits of a checksum. A reply packet 1205 may be returned by at least one of a node unit 107 during a reply period 1204. A reply packet 1205 may comprise two bytes 1206, wherein the first byte 1308 REPLY1 may be eight bits of a sixteen bit reply, and wherein the second byte 1309 REPLY2 may be the remaining eight bits of a sixteen bit reply. Additional reply bytes 1206 may be used for other purposes, for example, a CRC or other supporting functionality.

It will be helpful to one skilled in the art to consider that a significant driver of the maximum duration of a reply period 1204 and thus the quantity of reply bytes 1206 that may be sent during a reply period 1204 may be contingent on the capacity of a capacitor 615 which may power a microprocessor 605 during a reply period 1204, as regulator components 611 may be disabled during this period. A capacitor having a capacity of 470 microfarrad to 1000 microfarrad may be acceptable for one exemplary embodiment to reply with two bytes 1206 during a reply period 1205.

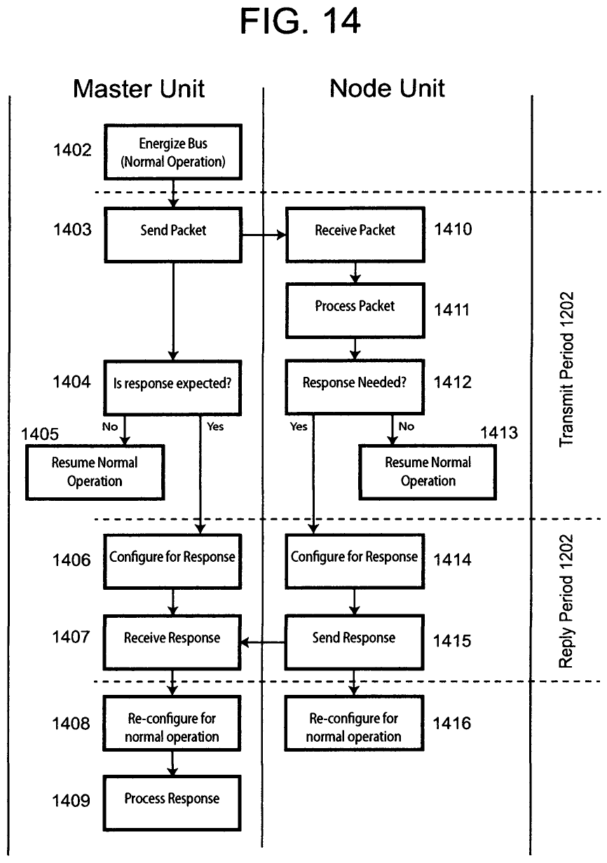

FIG. 14 may representatively illustrate a simplified flow chart of a communication with response between a master unit 102 and a node unit 107, according to various embodiments, and as may be controlled by a programming of a microprocessor, and may be described as follows.

A master unit 102 may energize a power control bus 112 for normal operation 1402. When a communication is needed and as may be directed by a master unit 102, a packet may be sent 1403 via a power control bus 112 via a variety of possible methods including those described above. A node unit 107 may receive said packet 1410. A node unit 107 may process a received packet 1411.

At this point or in response to a received packet 1411, a node unit may carry out a variety of actions. The action of a node unit may proceed if a checksum 1307 is correct, and if an address 1302, 1303 matches an address stored in a memory of a node unit 107, or if an address 1302, 1303 matches a broadcast address to which all node units 107 or a portion of node units 107 may be expected to respond to. An action of a node unit 107 may be based on the content of a command 1304, and may further be based on an argument 1305, 1306, or any combination thereof. For example, a command 1304 may indicate that a valve 109 should be operated, and an argument 1305, 1306, may indicate whether a valve 109 should be opened or shut. A programming of a microprocessor may cause a valve 109 to actuate in the desired directly by controlling signals 905, 906. A node unit may carry out any other action as well, for example but not limited to, depending on the exact embodiment and any accessory components, a node unit 107 may cause a fertilizer unit to begin dispensing fertilizer, or a node unit 107 may turn on or off lighting, indicating lighting, cameras, other environmental controls, air, sound, or movement so as to discourage wildlife from entering an area.

It may be a feature of a programming of a microprocessor that a determination may be made by one or more of a node unit 107, at step 1412, that a node unit 107 is expected to respond to a master unit 102. The determination of a response being expected may be based on the numerical value or range of values of a command 1304, or of the value or range of values of an argument 1305, 1306, or of any other bit or data that may be comprised within a packet 1203, or any combination or arrangement thereof.

In one exemplary embodiment, a command from a master unit 102, the command having a value of 0 to 128, may never expect a response from a node unit 107, and thus, at step 1412, and based on a command 1304, may resume normal operation and may not configure for a response. If a command from a master unit 102, the command having a value of 129 to 255, it may be assumed that a response is needed from a given node unit 107. If a response is needed from a node unit, based on the content of a command 1304 or other data comprised within a packet 1203, a node unit 107 or a portion of node units 107, or all node units 107 connected to the given power control bus 112 may configure for response 1414 which may be synchronized with a reply period 1202. A master unit 102 may determine in a similar way that a response is expected from a node unit 107 at step 1404 and may configure to receive a response at step 1406, which may also be synchronized with a reply period 1202. A node unit 107 may then respond with bits or bytes 1206, 1308, 1309, or response packet 1205, or any combination thereof at step 1415. A master unit 102 may receive the response at step 1407. After the response is communicated, a master unit 102 may re-configure for normal operation at step 1408 and may then process any response received at step 1409 if applicable. In a similar way, one or a portion of or all of a node unit 107 may re-configure for normal operation at step 1416.

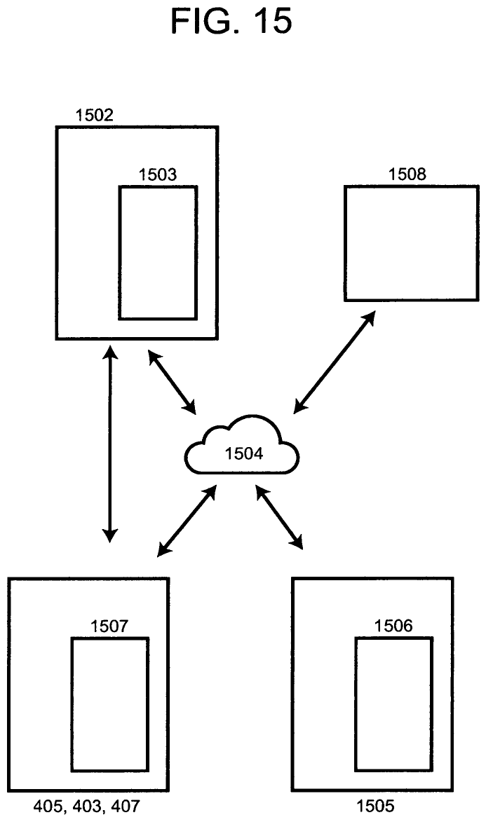

FIG. 15 may representatively illustrate a simplified flow chart of a communication between a master unit 102, a network or cloud 1504, a remote server 1505, and another device 1502 such as a smart phone, according to various embodiments. Various embodiments of communication and control may be utilized by any embodiment described herein.

A memory associated with a master unit 102 or one or more of a node unit 107 or any combination thereof may store via a volatile or non-volatile memory a watering program. A watering program 1507 may comprise a list of one or more of a scheduled event, for example, a time at which a node unit 107 should begin watering 114. A watering program 1507 may store other data as well, for example, a desired flow rate, pressure, or volume of liquid to be dispensed. A flow meter 204 may be used to measure the quantity of liquid dispensed. The duration of time a node unit 107 may operate a valve 109 to water 114 may be based at least partially on a value measured from a flow meter 204. A master unit may store a watering program 1507 in a memory of a microprocessor 405, supporting components 403, or radio communication device 407, or any combination thereof, the watering program may be emobodied via various methods as would be known to one skilled in the art of computer programming, and may use for example the CRON scheduling functionality of some Linux distributions, or may use a database, spread sheet, flat file, or other means of managing and scheduling from a list.

A master unit 102 may operate primarily from a watering program 1507 associated with the master unit 102. However, in various embodiments other methods of scheduling of watering or other events may be possible. For example, a radio communication device 407 may communicate directly with a device 1502 such as a smart phone, or may communicate with a cloud 1504, the internet, or any other network device, which may communicate with a server 1505, which may be remote. A smart phone or other device 1502 may also comprise a watering program 1503, or a user interface for setting, controlling, monitoring, or otherwise interacting with a watering program, or any combination thereof. A user may be able to cause a watering program 1503 to be at least partially transferred to a master unit 102, and wherein a master unit 102 may base at least a portion of watering program 1507 on at least a portion of a watering program 1503. In a similar way, a smart phone or other device 1502 may cause a watering program 1503 to be at least partially transferred to a server 1505, and wherein a server 1505 may base at least a portion of watering program 1506 on at least a portion of a watering program 1503. It is considered also that a user via user interface 1503 may remotely edit, monitor, or otherwise manage a watering program 1506 which may exist on a server 1505.

A master unit 102 may receive at least a portion of a watering program 1506 by a periodic query from master unit 102 to a server 1505, or alternately a server 1505 may push at least a portion of a watering program 1506 to a master unit 102 periodically, and wherein a master unit 102 may base at least a portion of watering program 1507 on at least a portion of a watering program 1506.

In various embodiments, any part of a watering program 1507, 1503, 1506, or any combination thereof may be at least partially based on a data received from another source 1508. Another source 1508 may be a sever or service which may be accessible via a network 1504 may comprise data that may be useful to a watering system, for example but not limited to, the weather and rain patterns in the local vicinity, temperature, humidity, wind, or any other useful data or any combination thereof in which any watering system described herein is to be operated.

Most of the equipment discussed above comprises hardware and associated software. We use the term software herein in its commonly understood sense to refer to programs or routines (subroutines, objects, plug-ins, etc.), as well as data, usable by a machine or processor. As is well known, computer programs generally comprise instructions that are stored in machine-readable or computer-readable storage media. Some embodiments of the present invention may include executable programs or instructions that are stored in machine-readable or computer-readable storage media, such as a digital memory. We do not imply that a “computer” in the conventional sense is required in any particular embodiment. For example, various processors, embedded or otherwise, may be used in equipment such as the components described herein.

Memory for storing software again is well known. In some embodiments, memory associated with a given processor may be stored in the same physical device as the processor (“on-board” memory); for example, RAM or FLASH memory disposed within an integrated circuit microprocessor or the like. In other examples, the memory comprises an independent device, such as an external disk drive, storage array, or portable FLASH key fob. In such cases, the memory becomes “associated” with the digital processor when the two are operatively coupled together, or in communication with each other, for example by an I/O port, network connection, etc. such that the processor can read a file stored on the memory. Associated memory may be “read only” by design (ROM) or by virtue of permission settings, or not. Other examples include but are not limited to WORM, EPROM, EEPROM, FLASH, etc. Those technologies often are implemented in solid state semiconductor devices. Other memories may comprise moving parts, such as a conventional rotating disk drive. All such memories are “machine readable” or “computer-readable” and may be used to store executable instructions for implementing the functions described herein.

A “software product” refers to a memory device in which a series of executable instructions are stored in a machine-readable form so that a suitable machine or processor, with appropriate access to the software product, can execute the instructions to carry out a process implemented by the instructions. Software products are sometimes used to distribute software. Any type of machine-readable memory, including without limitation those summarized above, may be used to make a software product. That said, it is also known that software can be distributed via electronic transmission (“download”), in which case there typically will be a corresponding software product at the transmitting end of the transmission, or the receiving end, or both.

In view of the many possible embodiments to which the principles of the disclosed technology may be applied, it should be recognized that the illustrated embodiments are only preferred examples and should not be taken as limiting the scope of the disclosure. We claim as our invention all that comes within the scope and spirit of the appended claims.

1) A watering system, comprising:
at least one master unit and at least one node unit;
wherein the at least one master unit and at least one node unit are electrically connected via at least one power control bus;
wherein the at least one master unit is configured to send at least one of a first byte transmission to the at least one node unit via a power control bus; and
wherein the at least one node unit is configured to send at least one of a response byte to the at least one master unit via at least one of a power control bus.

2) The watering system of claim 1,
wherein at least one of a first byte transmission comprises at least a first command;
wherein the at least one node unit is configured to be responsive to the at least a first command;
wherein the at least one of a node unit is configure to send the at least one of a response byte in response to the at least a first command.

3) The watering system of claim 2, wherein at least one portion of a power control bus comprises coaxial cable.

4) The watering system of claim 1,
wherein the at least one master unit comprises at least one of a switching element electrically connected to the at least one power control bus;
wherein the at least one switching element is operably coupled to a first microprocessor of the at least one master unit;
wherein the at least one master unit is configured to transmit at least one of a transmit byte via a power control bus in response to controlling at least one switching element by the first microprocessor.

5) The watering system of claim 4,
wherein the at least one node unit comprises at least one current sink electrically connected to the at least one power control bus;
wherein the at least one current sink is operably coupled to a second microprocessor of the at least one node unit;
wherein the at least one node unit is configured to send at least one of a response byte via a power control bus in response to controlling at least one current sink by the second microprocessor.

6) The watering system of claim 5,
wherein the at least one node unit comprises at least one of a valve control circuitry;
wherein at least one of a valve control circuitry is operably connected to the a second microprocessor of the at least one node unit;
wherein at least one of a valve is electrically connected to at least one of a valve control circuitry;
wherein the a second microprocessor of the at least one node unit is configured to operate the at least one valve via the at least one valve control circuitry in response to the at least of of a transmit byte.

7) The watering system of claim 5,
wherein the at least one node unit is electrically connected to at least one of a flow meter device;
wherein the at least one of a response byte comprises data representative of a measurement of the at least one of a flow meter device.

8) The watering system of claim 5,
wherein the at least one node unit is electrically connected to at least one of a moisture sensor;
wherein the at least one of a response byte comprises data representative of a measurement of the at least one of a moisture sensor.

9) A method of communicating data between nodes of a watering system, the method comprising:
at least one of a master unit transmitting at least one of a packet via a power control bus during a transmit period;
at least one of a node unit receiving the at least one of a packet via a power control bus during a transmit period;
at least one of a node unit sending at least one of a response via a power control bus during a reply period.

10) The method of claim 9, the method further comprising:
during at least one portion of a reply period, at least one of a master unit changing configuration of at least one portion of a circuitry of the at least one of a master unit.

11) The method of claim 10,
wherein the changing configuration of at least one portion of a circuitry of the at least one of a master unit includes enabling at least one of a weak pull up component,
wherein the at least one of a weak pull up component is electrically connected to a power control bus.

12) The method of claim 9, the method further comprising:
during at least one portion of a reply period, at least one of a microprocessor operably coupled to the at least one of a node unit configuring a circuitry of the at least one of a node unit to enter a lower power state.

13) The method of claim 10, the method further comprising:
at least one of a program of at least one of a microprocessor operably coupled to the at least one of a master unit making a determination to change configuration based on at least one of a command, wherein the at least one of a command is comprised within at least one of a packet transmitted via a power control bus.

14) The method of claim 12, the method further comprising:
at least one of a program of at least one of a microprocessor operably coupled to the at least one of a node unit making a determination to configure a circuitry of the at least one of a node unit to enter a lower power state is based on at least one of a command, wherein the at least one of a command is comprised within at least one of a packet transmitted via a power control bus.

15) A packet for communicating data in a watering system, the packet comprising:
a transmit packet comprising six eight-bit bytes, the transmit packet comprising at least one of a node address, at least one of a command, at least one of an argument, and at least one of a checksum;
a reply packet comprising at least one eight-bit byte.

16) The packet of claim 15, wherein
a reply packet is generated during a substantially predictable interval of time following a transmit packet.

17) The packet of claim 16, wherein
the generation of a reply packet or the non-generation of a reply packet is substantially predictable based on at least one of a command of a transmit packet.

18) The packet of claim 17, wherein
the at least one byte of a reply packet is based on at least one of a command of a transmit packet.

19) The packet of claim 18, wherein
the at least one byte of a reply packet is based on reading at least one flow meter device.

20) The packet of claim 18, wherein
the at least one byte of a reply packet is based on reading at least one moisture sensor.
